(19) United States
(12) Patent Application Publication
(10) Pub. No.: US 2017/0296375 A1
(43) Pub. Date: Oct. 19, 2017

(54) Urine Capturing System

(71) Applicant: Rex Kramer, Crystal, MI (US)

(72) Inventor: Rex Kramer, Crystal, MI (US)

(21) Appl. No.: 15/131,651

(22) Filed: Apr. 18, 2016

(51) Int. Cl. A61F 5/453; A61F 5/44

(52) U.S. Cl. CPC A61F 5/453; A61F 5/4408

(57) ABSTRACT

A urine capturing system for capturing leaked urine from a penis includes an article of clothing that may be worn thereby facilitating the article of clothing to cover genitals. A sheath is provided and the sheath may be selectively worn on a penis. The sheath is comprised of a fluid impermeable material. The sheath inhibits urine leaking outwardly of the penis from contacting the article of clothing. Thus, the sheath inhibits the article of clothing from becoming visibly wet from the leaked urine.

### BACKGROUND OF THE DISCLOSURE

### Field of the Disclosure

The disclosure relates to capturing devices and more particularly pertains to a new capturing device for capturing urine leaked from a penis.

### SUMMARY OF THE DISCLOSURE

An embodiment of the disclosure meets the needs presented above by generally comprising an article of clothing that may be worn thereby facilitating the article of clothing to cover genitals. A sheath is provided and the sheath may be selectively worn on a penis. The sheath is comprised of a fluid impermeable material. The sheath inhibits urine leaking outwardly of the penis from contacting the article of clothing. Thus, the sheath inhibits the article of clothing from becoming visibly wet from the leaked urine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a urine capturing system according to an embodiment of the disclosure.

FIG. 2 is a back view of an embodiment of the disclosure.

FIG. 3 is a cross sectional view taken along line 3-3 of FIG. 1 of an embodiment of the disclosure.

FIG. 4 is a top view of an embodiment of the disclosure.

FIG. 5 is a perspective in-use view of an embodiment of the disclosure.

### DESCRIPTION OF THE PREFERRED EMBODIMENT

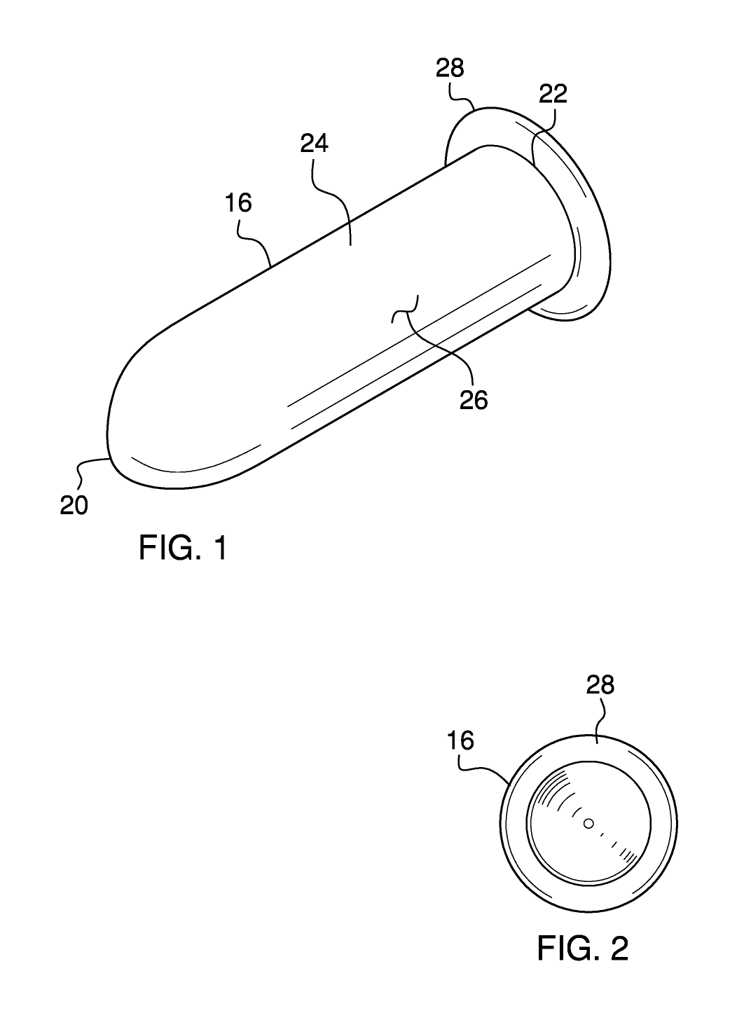

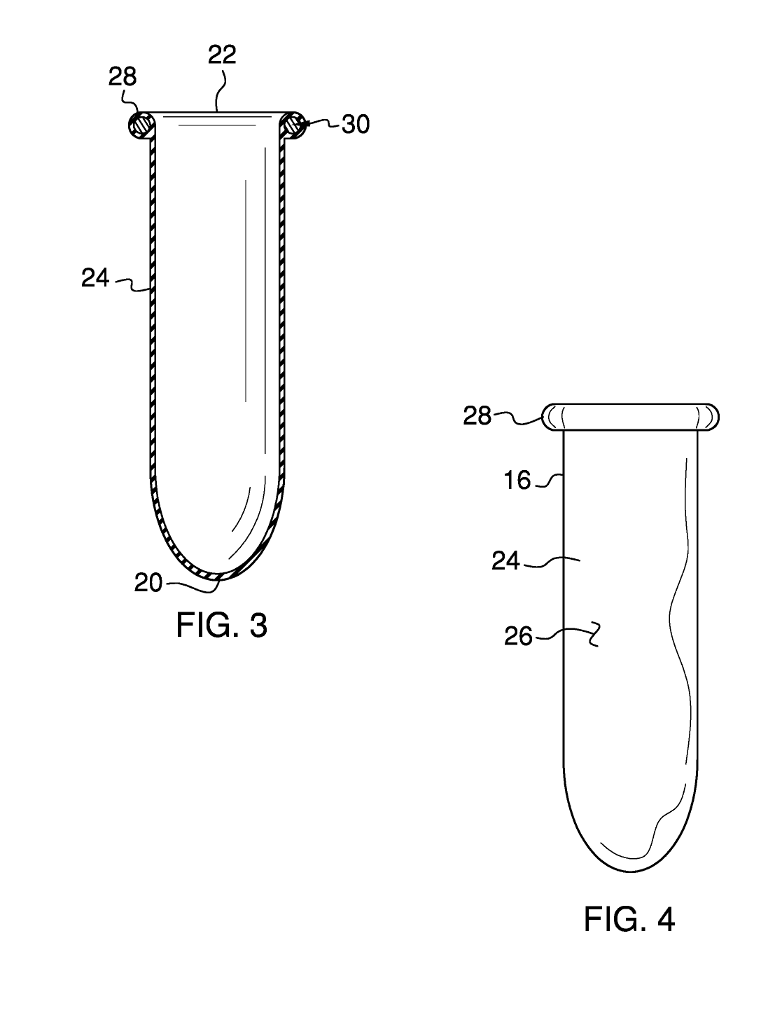

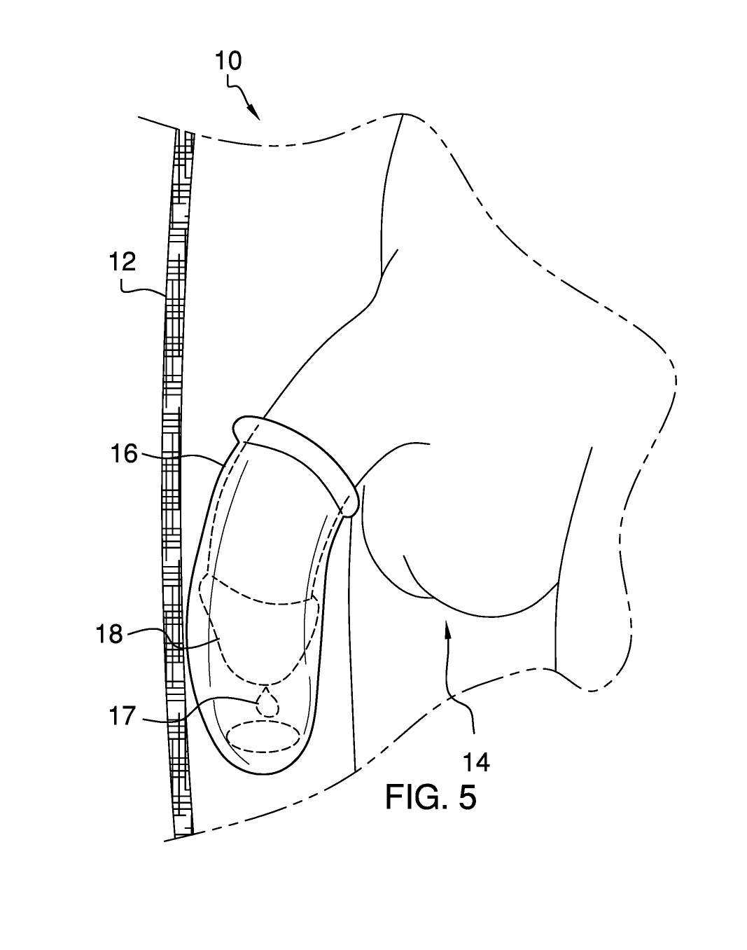

With reference now to the drawings, and in particular to FIGS. 1 through 5 thereof, a new capturing device embodying the principles and concepts of an embodiment of the disclosure and generally designated by the reference numeral 10 will be described.

As best illustrated in FIGS. 1 through 5, the urine capturing system 10 generally comprises an article of clothing 12. The article of clothing 12 may be worn thereby facilitating the article of clothing to cover genitals 14. The article of clothing 12 may comprise pants, shorts or other article of clothing worn to cover the genitals 14. Moreover, the article of clothing 12 may be comprised of a fluid absorbent material such as cotton or the like.

A sheath 16 is provided and the sheath 16 may be selectively worn on a penis 18. The sheath 16 is comprised of a fluid impermeable material. Additionally, the sheath 16 is comprised of a non-latex material. The sheath 16 inhibits urine 17 leaking outwardly of the penis 18 from contacting the article of clothing 12. Thus, the sheath 16 inhibits the article of clothing 12 from becoming visibly wet.

The sheath 16 has a first end 20, a second end 22 and an outer wall 24 extending therebetween. The outer wall 24 is continuous such that the sheath 16 has a cylindrical shape and the outer wall 24 has an outer surface 26. The sheath 16 is substantially hollow and the second end 22 is open. Thus, the second end 22 may insertably receive the penis 18. Moreover, the sheath 16 has a length sufficient to substantially cover the penis 18 when the sheath 16 is worn on the penis 18.

An elastic member 28 is provided and the elastic member 28 coupled to the sheath 16. The elastic member 28 compresses the sheath 16 around the penis 18 when the sheath 16 is selectively worn on the penis 18. The elastic member 28 is coupled to the outer wall 24 and the elastic member 28 is positioned on the outer surface 26 of the sheath 16. The elastic member 28 is coextensive with the second end 22. Thus, the elastic member 28 inhibits the urine 17 from escaping from the sheath 16 when the sheath 16 is worn on the penis 18.

The second end 22 of the sheath 16 may be folded down and attached to the outer surface 26 of the sheath 16. Thus, a channel 30 may be formed. The channel 30 may extend completely around the sheath 16. The elastic member 28 may be positioned within the channel 30.

In use, the article of clothing 12 is worn in the convention of pants and shorts. The sheath 16 is carried when a user is attending an event involving remote, public bathrooms. The sheath 16 is worn on the penis 18 prior to urinating. Thus, the sheath 16 captures urine 17 that leaks from the penis 18 prior to urination. The sheath 16 inhibits the leaked urine 17 from contacting the article of clothing 12. Thus, the article of clothing 12 is inhibited from becoming visibly wet with urine 17 thereby facilitating peace of mind for the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. A urine capturing system comprising:
an article of clothing being configured to be worn thereby facilitating said article of clothing to cover genitals;
a sheath being configured to be selectively worn on a penis, said sheath being comprised of a fluid impermeable material wherein said sheath is configured to inhibit urine leaking outwardly of the penis from contacting said article of clothing thereby inhibiting said article of clothing from becoming visibly wet with the leaked urine.

2. The assembly according to claim 1, wherein said sheath has a first end, a second end and an outer wall extending therebetween, said outer wall being continuous such that said sheath has a cylindrical shape, said sheath being substantially hollow, said second end being open wherein said second end is configured to insertably receive the penis.

3. The assembly according to claim 1, further comprising an elastic member being coupled to said sheath wherein said elastic member is configured to compress said sheath around the penis when said sheath is selectively worn on the penis.

4. The assembly according to claim 3, wherein:
said sheath has an outer wall and a second end; and
said elastic member is coupled to said outer wall, said elastic member being coextensive with said second end wherein said elastic member is configured to inhibit the urine from escaping from said sheath when said sheath is worn on the penis.

5. A urine capturing system comprising:
an article of clothing being configured to be worn thereby facilitating said article of clothing to cover genitals;
a sheath being configured to be selectively worn on a penis, said sheath being comprised of a fluid impermeable material wherein said sheath is configured to inhibit urine leaking outwardly of the penis from contacting said article of clothing thereby inhibiting said article of clothing from becoming visibly wet with the leaked urine, said sheath having a first end, a second end and an outer wall extending therebetween, said outer wall being continuous such that said sheath has a cylindrical shape, said sheath being substantially hollow, said second end being open wherein said second end is configured to insertably receive the penis; and
an elastic member being coupled to said sheath wherein said elastic member is configured to compress said sheath around the penis when said sheath is selectively worn on the penis, said elastic member being coupled to said outer wall, said elastic member being coextensive with said second end wherein said elastic member is configured to inhibit the urine from escaping from said sheath when said sheath is worn on the penis.
